(19) United States
(12) Patent Application Publication
(10) Pub. No.: US 2016/0279229 A9
(43) Pub. Date: Sep. 29, 2016

(54) Viral Vaccine Vectors

(71) Applicant: YALE UNIVERSITY, NEW HAVEN, CT (US)

(72) Inventor: John K. Rose, Guilford, CT (US)

(21) Appl. No.: 14/740,979

(22) Filed: Jun. 16, 2015

Related U.S. Application Data
(62) Division of application No. 12/747,591, filed on Aug. 16, 2010, now abandoned.
(60) Provisional application No. 61/010,889, filed on Jan. 11, 2008.

(51) Int. Cl. A61K 39/21

(52) U.S. Cl. CPC A61K 39/21; A61K 2039/5256

(57) ABSTRACT

The present invention relates to a hybrid-viral vector system, in particular, but not exclusively, to a hybrid-viral vector system that can be used as a vaccine vector.

### CROSS-REFERENCE TO RELATED APPLICATIONS

The present application is a divisional of U.S. patent application Ser. No. 12/747,591, which is U.S. National Stage Application of International Application PCT/IB09/50110 filed on Jan. 12, 2009, which claims priority pursuant to 35 U.S.C. §119(e) of U.S. Provisional Application No. 61/010,889 filed Jan. 11, 2008, each of which are hereby incorporated by reference in their entirety herein.

### FIELD OF THE INVENTION

The present invention relates to a hybrid-viral vector system, in particular, but not exclusively, to a hybrid-viral vector system that can be used as a vaccine vector.

### BACKGROUND OF THE INVENTION

Enveloped RNA viruses typically have a highly organized structure. At the center of the virus particle is a nucleocapsid core containing RNA bound to one or more nucleocapsid proteins. Matrix proteins are often organized between the core and the external membrane envelope, which contains membrane-spanning glycoproteins. One or more glycoproteins may be required for virus binding to receptors on the cell surface and for catalyzing fusion of the viral membrane with the plasma membrane or with endosomal membranes after virus uptake.

Vesicular stomatitis virus (VSV) is a rhabdovirus that contains a single negative-stranded genomic RNA that is transcribed by a virion polymerase into five mRNAs encoding five structural proteins. The nucleocapsid protein encapsidates the RNA genome, two proteins form the polymerase complex bound to the nucleocapsid, a matrix protein is associated with the nucleocapsid and the membrane, and a single (transmembrane) envelope spike glycoprotein (G) extends from the viral envelope. The G protein functions to bind virus to a cellular receptor and to catalyze fusion of the viral membrane with cellular membranes to initiate the infectious cycle.

RNA replicons from alphaviruses including Semliki Forest virus (SFV) have been developed and used for transient expression of foreign proteins in mammalian cells and also as experimental vaccine vectors (1-4). The alphavirus genome is a capped and polyadenylated positive-strand RNA molecule about 12 kb in length. The genomic RNA itself is an mRNA that encodes the viral replicase. A subgenomic mRNA copied from the antigenomic RNA following replication encodes the alphavirus structural proteins. RNA transcribed from SFV cDNA can initiate viral RNA replication following transfection into cells (5).

The present inventors have previously tested an SFV replicon developed by Liljestrom and Garoff (5) for expression of the vesicular stomatitis virus (VSV) glycoprotein (G) (6). The starting SFV RNA replicon was derived from a DNA copy of SFV from which the genes for the SFV structural proteins were removed. The VSV G gene was inserted in place of genes encoding the SFV structural proteins. When this so-called ‘SFVG’ replicon RNA expressing only the SFV replication proteins and VSV G protein was transfected into BHK-21 cells, it initially replicated in a small fraction of transfected cells. The inventors also found that it produced infectious, low density, membrane-enveloped particles that budded from the cells and infected and killed all cells in the culture within 2 to 3 days. These infectious particles could be propagated (passaged) indefinitely in tissue culture and their infectivity was inactivated by a VSV neutralizing antibody which binds the VSV G protein (6, 9). Although the precise mechanism of generation of the SFVG infectious particles remains unknown, it is believed to involve release of vesicles containing VSV G protein and SFV RNA. The replication of all positive-strand RNA viruses including SFV occurs in association with cellular membranes (10). SFV replication occurs in association with cytopathic vacuoles containing invaginations called spherules, which are probably the sites of SFV RNA synthesis (11-13). These spherules are also seen on the surface of SFV-infected cells (12) and could be precursors involved in formation of the infectious particles containing VSV G (6). Experimental SFV particle-based vaccines are normally derived from a complementation/packaging system in which SFV replicons encoding foreign antigenic proteins are packaged into SFV-like particles by SFV structural proteins expressed in trans (5). Such a complementation system has traditionally been required for alphavirus vector systems because of the strict size limit for encapsidation of viral genomic RNA. Unless the structural genes are deleted, there is no space for inclusion of genes expressing foreign antigens. Because these complemented particles do not encode SFV structural proteins, they replicate for only a single cycle when inoculated into animals. This system is a relatively complex one, and the resulting single cycle replication can lead to potential problems with providing an efficient vaccine.

There is therefore a need in the art for further alphavirus hybrid-vector vaccine systems.

### SUMMARY OF THE INVENTION

The present inventors have surprisingly discovered that hybrid-viral vector systems can unexpectedly elicit strong humoral and cell mediated immune responses, with the cell mediated response being directed against a protein expressed by the hybrid-viral vector vaccine system. Additionally, the humoral immune response is directed against a protein expressed by the hybrid-viral vector system which may or may not be the same as the protein against which the cell mediated immune response is targeted.

Accordingly, in one aspect, the present invention relates to a hybrid-virus vector vaccine comprising a nucleotide sequence encoding: alphavirus non-structural protein nucleotide sequences; a first nucleotide sequence being a viral structural nucleotide sequence, said sequence not being an alphavirus structural gene sequence; and a second nucleotide sequence, wherein said second nucleotide sequence encodes a heterologous antigenic protein of interest; and wherein the vector lacks a functional nucleotide sequences which encode alphavirus structural proteins.

As herein defined, a “heterologous antigen” is any antigen which is not normally expressed by the viral vector and which, when expressed by the vector, is suitable to have an immune response mounted there against.

As herein defined, an alphavirus non-structural protein can be selected from the group consisting of nsp1, nsp2, nsp3 and nsp4.

As defined herein, an alphavirus structural protein can be selected from the group consisting of an alphavirus capsid protein and at least one spike protein.

Without wishing to be bound by theory, the inventor has identified that transfection of the hybrid-viral vector vaccine advantageously and unexpectedly results in the production of infectious virus like particles (VLPs) encoding the second nucleotide sequence which encode the heterologous antigen and which induce strong humoral and cell mediated immune responses targeted against the heterologous antigen.

In certain embodiments, the hybrid-viral vector vaccine first structural nucleotide sequence comprises a vesiculovirus or rhabdovirus surface glycoprotein. In certain embodiments, the surface glycoprotein is vesicular stomatitis virus glycoprotein (VSVG) protein.

In certain embodiments, the first structural nucleotide sequence can be operably linked to the alphavirus subgenomic promoter.

In certain embodiments, the hybrid-viral vector vaccine alphavirus nucleotide sequences are Semliki forest virus (SFV) nucleotide sequences.

In certain embodiments, the hybrid-vector vaccine may be a DNA vector, a cDNA vector or a transcript thereof. Typically, the transcript is capped and polyadenylated. In certain embodiments, the hybrid-viral vector vaccine further comprises an inducible non-alphavirus promoter or promoter-like sequence or promoter element. Optionally, the non alphavirus promoter drives expression of at least the alphavirus non structural proteins. In certain embodiments, the non-alphavirus promoter is the cytomegalovirus (CMV) immediate early promoter. The promoter that drives expression of the alphavirus RNA can be any promoter having a DNA sequence recognized by a DNA-dependent RNA polymerase, and can be any effective promoter/DNA-dependent RNA polymerase combination (viral or cellular), preferably one that is efficient. The skilled person is well aware of the number of possible promoter/polymerase combinations that would be acceptable to use.

In certain embodiments, the hybrid-viral vector vaccine may further include a third nucleotide sequence encoding a further heterologous antigenic protein of interest. In certain embodiments, the second nucleotide sequence is a heterologous antigenic peptide which is expressed on the surface of, or is secreted by an infectious agent. In certain embodiments, the second nucleotide sequence encodes a viral protein or fragment thereof, e.g. an SIV or HIV protein or protein fragment.

In certain embodiments, the hybrid-viral vector vaccine is selected from the group consisting of; pSFV1-Gdp (or pSFVdpG-X) and pCMVSFV-Gdp, or transcripts thereof. In certain further aspects, the vector or virus like particle generated from said vector is non-pathogenic to a cell or animal transfected with said vector or infected with said virus like particle.

In certain further aspects, the present invention relates to a composition including a hybrid-viral vector vaccine or virus like particle generated from said vector. In certain further aspects, the present invention relates to a pharmaceutical composition comprising a hybrid-viral vector vaccine as hereinbefore defined, or to a virus like particle generated from said vector along with at least one carrier, diluent or excipient. In certain embodiments, the pharmaceutical composition may further comprise, or is administered along with at least one adjuvant. Suitable adjuvants include, but are not limited to the group consisting of Freund's complete adjuvant, Freund's incomplete adjuvant, Quil A, Detox, ISCOMs and squalene.

In a further aspect, the present invention relates to the use of a hybrid-viral vector vaccine, or a virus like particle generated from said vector, to elicit an immune response in a mammal, said immune response being directed to a gene product encoded by the first, second or third nucleotide sequence of the hybrid-viral vector vaccine. Typically, said immune response is both a cell mediated immune response and a humoral immune response.

Optionally, the hybrid-viral vaccine vector, or a virus like particle generated from said vector, elicits a cell mediated response in an animal, preferably in a mammal. Typically the cell mediated response is accompanied by the generation of a humoral response, wherein said antibodies have binding specificity for an antigen expressed by the hybrid virus vector. Typically, the antibodies produced as a result of the humoral immune response neutralise infectious agents expressing the heterologous antigen encoded by the hybrid-viral vaccine vector. In certain embodiments, the antibodies neutralise an infectious agent expressing the antigen expressed from the second nucleotide sequence.

In certain embodiments, the antibodies have binding specificity to vesiculovirus or rhabdovirus, e.g. VSV neutralising antibodies.

The term “specifically binds”, “selectively binds” or “binding specificity” refers to the ability of the humanised antibodies or binding compounds of the invention to bind to a target epitope present on VSV with a greater affinity than that which results when bound to a non-target epitope. In certain embodiments, specific binding refers to binding to a target with an affinity that is at least 10, 50, 100, 250, 500, or 1000 times greater than the affinity for a non-target epitope. In certain embodiments, this affinity is determined by an affinity ELISA assay. In certain embodiments, affinity is determined by a BIAcore assay. In certain embodiments, affinity is determined by a kinetic method. In certain embodiments, affinity is determined by an equilibrium/solution method.

In certain embodiments, the neutralising antibodies provide at least 70% protection, preferably about 100% protection (i.e. substantially total protection) against pathogenesis, disease or death induced by the infectious agent upon challenge by the infectious agent expressing the antigenic component.

In certain embodiments, the cell mediated immune response includes the generation of a T cell response specific to the antigen encoded by the second nucleotide sequence. Typically, the T cell mediated response comprises the production of CD8+ T cells and optionally IL-17 producing T cells (Th17 T cells). Typically, said T cells are directed to the heterologous antigen encoded by the second nucleotide sequence. In a further embodiment, the cell mediated immune response includes the generation of memory T cells which are specific to a heterologous antigen encoded by the hybrid-virus vector.

In certain embodiments, the memory T cell response may be recalled following boosting of the immune response via further administration of an effective dose of the hybrid-vector vaccine system or VLP generated from said vector.

In certain embodiments, the hybrid-viral vector encodes, or the VLP generated from said vector contains, a retroviral protein or fragment thereof, and wherein the CD8+ T-cell response is specific to the retroviral protein or fragment thereof. In certain embodiments, the retroviral protein or a fragment thereof is a gag or env protein or protein fragment. In certain embodiments, the retroviral proteins are derived from HIV (human immunodeficiency virus-1, or human immunodeficiency virus-2) or SIV (simian immunodeficiency virus). In a yet further aspect, the present invention relates to a method of treating and/or preventing disease in a subject, the method comprising the steps of: providing a therapeutically effective amount of a hybrid viral vector vaccine or a virus like particle generated from said vector as described herein, and administering the same to a subject in need of such treatment.

As used herein, the term “effective amount” or “therapeutically effective amount” means the amount of the hybrid viral vector vaccine or a virus like particle generated from said vector of the invention which is required to prevent the particular disease condition, or which reduces the severity of and/or ameliorates the disease condition or at least one symptom thereof or condition associated therewith.

In order to prevent a disease, a prophylactically effective amount of the hybrid viral vector vaccine or a virus like particle generated from said vector is used. The term “prophylactically effective amount” relates to the amount of a composition which is required to prevent the initial onset, progression or recurrence of a disease condition, or at least one symptom thereof in a subject following the administration of the compounds of the present invention.

As used herein, the term “treatment” and associated terms such as “treat” and “treating” means the reduction of the progression, severity and/or duration of a disease condition or at least one symptom thereof. The term ‘treatment’ therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition.

As used herein, the term “subject” refers to an animal, preferably a mammal and in particular a human. In a particular embodiment, the subject is a mammal, in particular a human. The term “subject” is interchangeable with the term “patient” as used herein.

In certain embodiments, the disease is a malignant disease, such as a cancer, or an infectious disease such as a disease caused by a virus, a bacterium, a fungus or a protozoon. In certain embodiments the vector, or virus like particle generated from said vector, is administered in combination with an adjuvant. Suitable adjuvants include, but are not limited to the group consisting of Freund's complete adjuvant, Freund's incomplete adjuvant, Quil A, Detox, ISCOMs and squalene.

In certain further aspect, the present invention relates to a method for vaccinating a subject, wherein the method comprises the step of administering a pharmaceutical acceptable quantity of a hybrid viral vector vaccine of the present invention, or a virus like particle generated from said vector to a subject in need of such treatment, wherein administration of the vector or the virus like particle is sufficient to elicit an immune response in the subject. In certain embodiments, the vaccine may be administered as a prophylactic vaccine or as a therapeutic vaccine.

In a further aspect, the present invention provides a method of mediating a memory T cell immune response in a subject to a heterologous antigen following the initial administration of a vector of the present invention, or a virus like particle generated from said vector which encode a heterologous antigen, the method including the steps of: administering a vaccine vector or VLP generated from said vector to a subject in an amount which is effective to elicit an immune response in the subject, said vector being described hereinbefore; administering a second effective amount of the vaccine vector or a VLP derived therefrom at a second, subsequent time period, wherein step b) causes an immune response to be mediated by memory T cells which are effective against the heterologous antigenic component of the vaccine vector or VLP.

In certain embodiments, the second effective amount of the vaccine is administered to the subject at a time point when memory cells to the antigen delivered from the primary vaccine are present in the subject, which may be any time from a few days, preferably from at least one week, or at least three weeks, up to a number of years after the primary vaccination, for example at least 10 years following the administration of the initial administered amount. Again, with regard to the “maximal” time, as long as memory cells are present in the subject, they will expand even years after a primary vaccination.

In a yet further aspect, the present invention provides the use of the hybrid viral vector or a virus like particle generated from a vector of the present invention in the preparation of a medicament for the treatment of an infectious disease.

A yet further aspect of the present invention provides a hybrid viral vector or a virus like particle generated from a vector of the present invention for use in treating an infectious disease.

In certain embodiments, the infectious disease is a disease resulting from an infectious agent such as a virus, bacteria, fungi or protozoa. In certain embodiments the infectious disease results from a viral infection where the virus is selected from the group comprising but not limited to human immunodeficiency virus (HIV), hepatitis A virus (HAV), hepatitis B (HBV), hepatitis C (HCV), any other hepatitis-associated virus, human papillomavirus (HPV) and especially high-risk oncogenic human papillomavirus types, Kaposi's Sarcoma-Associated Herpesvirus (KSHV) (also known as Human Herpesvirus-8 (HHV-8)), Herpes Simplex virus (HSV) (any subtype), Respiratory Syncytial Virus (RSV) and associated respiratory viruses, Influenza viruses, coronaviruses including SARS-associated Coronavirus (SARS-CoV), rhinovirus, adenovirus, SIV, rotavirus, human papilloma virus, arbovirus, measles virus, polio virus, rubella virus, mumps virus, papova virus, cytomegalovirus, varicella-zoster virus, varicella virus, huntavirus and any emergent virus, in particular Ebola virus, Marburg virus, West Nile virus (WNV), St Louis Encephalitis virus (SLEV), Rift Valley Fever virus (RVFV) and other members of the Bunyaviridae.

In certain embodiments, the infectious disease results from a bacterial infection, wherein the bacterium is selected from the group comprising but not limited to Escherichia, Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma.

In a yet further aspect, the present invention provides the use of the hybrid viral vector or a virus like particle generated from a vector of the present invention in the preparation of a medicament for the treatment of cancer.

A yet further aspect of the present invention provides a hybrid viral vector or a virus like particle generated from a vector of the present invention for use in treating a cancerous or malignant condition.

In certain embodiments, the cancerous or malignant condition may include a condition selected from the group comprising, but not limited to: malignant melanoma, chronic myelogenous leukaemia, hairy cell leukaemia, multiple myeloma, renal cell carcinoma, hepatocellular carcinoma, colorectal cancer, gastric cancer, head and neck cancer, osteosarcoma, breast cancer, ovarian cancer, cervical cancer, prostate cancer, Non-Hodgkins lymphoma.

In a yet further aspect, the present invention relates to a protein expression system including DNA hybrid-vector comprising: alphavirus non-structural proteins nucleotide sequences; a first nucleotide sequence being a viral structural nucleotide sequence, said sequence being a non-alphavirus structural nucleotide sequence; and

a second nucleotide sequence, wherein the second nucleotide sequence is capable of expressing a protein of interest, wherein the vector lacks a functional nucleotide sequence which encodes a structural protein of the alphavirus.

In certain further aspects, the present invention relates to a method of expressing at least one protein of interest in a cell comprising the steps of: transfecting a cell population with a vector of the present invention; allowing expression of the nucleotide sequence of interest within the cell culture; harvesting the cell population; and purifying the protein of interest. Further features and advantages of the present invention will be apparent from the attached claims and description.

### BRIEF DESCRIPTION OF THE FIGURES

FIG. 1 shows that replication is required for induction of neutralizing antibody by SFVG particles. Mice were inoculated i.m. (intra-muscularly) with 6*10³ infectious units (i.u.) of SFVG untreated or treated with 100 ml ultraviolet light (UV), or with 10⁵ i.u. of SFVG or 10⁵ pfu (plaque forming units) of VSV as indicated. Pooled sera from mice were assayed for neutralizing titers to VSV on day 28 post-inoculation. UV (ultraviolet) inactivation of SFVG particles before inoculation abolished generation of anti-VSV G neutralizing antibody. The neutralizing antibody titer to VSV in sera from mice inoculated with (10⁵ i.u.) of SFVG was equivalent to that in sera from mice inoculated with 10⁵ pfu of VSV (1:5,120).

FIG. 2 shows that vaccination with SFVG particles protects mice against pathogenesis caused by wild-type VSV. Twelve BALB/c mice were immunized with 5×10⁵ i.u. of SFVG particles by i.m. injection. At 36 days post-immunization, these mice were challenged with 5*10⁷ pfu of wild-type VSV by the intravenous route. Twelve non-immunized BALB/c mice were challenged as controls. Following challenge, mice were weighed daily for up to 14 days and observed for signs of pathogenesis. Any animal exhibiting paralysis or distress during this period was euthanized. The graph shows the average weights of the mice±one standard deviation. Numbers above the x-axis indicate the number of mice in the control group that died on the corresponding day.

FIGS. 3A-3D are series of images that shows co-expression of VSVG protein and gp140 in cells infected with SFVG-gp140 particles. FIG. 3A) Indirect immunofluorescence microscopy of BHK-21 cells infected with SFVG-gp140 particles for 20 hours, fixed, and stained by using antibodies recognizing VSV G protein on the cell surface followed by an ALEXA FLUOR® 594 secondary antibody. FIG. 3B) The same field of cells permeabilized and stained by using sheep anti-gp120 antibody followed by FITC-conjugated secondary antibody. FIG. 3C) Merge of FIG. 3A and FIG. 3B. FIG. 3D) Differential interference contrast (DIC) image of cells with uninfected cells noted by white arrows.

FIG. 4 shows protein expression by SFVG and SFVG-gp140. BHK-21 cells were infected with SFVG particles, with SFVG-gp140 particles, with a VSV recombinant expressing gp140, or left uninfected. Metabolic labeling with [³⁵5]-methionine was between 5 and 6 hours post-infection. Cell lysates were prepared and either run directly on a 10% PAGE (VSVgp140) or immunoprecipitated using antibodies to VSV or HIV Env as indicated.

FIGS. 5A-5B are series of graphs that shows that SFVG-gp140 vaccination generates primary, Env-specific CD8+ T cell responses that are readily recalled upon boosting. FIG. 5A (upper panels) shows representative FACS plots of CD8+ T-cells from spleens of individual BALB/c mice inoculated i.m. with 10⁵ pfu of SFVG or SFVG-gp140 and analyzed at 7 days post-inoculation. CD8+, Env tetramer⁺, and CD62L¹⁰ cells are in the upper left quadrants (0.035% SFVG and 2.21% SFVG-gp140). FIG. 5A (lower panels) show the same analysis done on CD8⁺ T cells from individual mice inoculated with SFVG or SFVG-gp140, boosted at day 29 with vPE16, and then analyzed at day 35 (0.088% 3.9% SFVG+boost and 21.0% SFVG-gp140+boost). FIG. 5B shows the average CD8⁺, Env tetramer⁺ and CD62L¹⁰ cells from multiple mice primed or primed and boosted as in A (N=4-5 for all groups except SFVG prime group only where N=2). Background responses (CD8+, Env tetramer⁺ and CD62L¹⁰) determined from VSV vaccinated mice were subtracted and were <0.06%. Error bars represent one standard deviation.

FIG. 6 shows a pSFV1-Gdp vector map. The pSFV1-Gdp vector map shows the positions of the SFV non-structural protein genes, VSV G, and the two SFV subgenomic promoters (arrowheads). The second is followed by three unique cloning sites. The Spe I site is used to linearise the DNA prior to in vitro transcription. The selectable AmpR marker is also shown.

FIG. 7 shows a pCMVSFV-Gdp vector map. The pCMVSFV-Gdp vector map shows the positions of the CMV promoter, SFV nonstructural protein genes, VSV G, and the two SFV subgenomic promoters (arrowheads). The second is followed by two unique cloning sites. This DNA launched vector is transfected directly onto cells to derive the infectious particles.

### DETAILED DESCRIPTION OF THE INVENTION

The immunogenicity of SFVG particles has not previously been tested in an animal model. As detailed below, the present inventors have examined the potential of these particles as a vaccine vector in a mouse model. The present inventors surprisingly discovered that the particles unexpectedly induced a potent neutralizing antibody response to VSV in mice. Mice vaccinated with these particles were protected from all weight loss and from a lethal encephalitis caused by a high dose of wild-type VSV given intravenously.

The present inventors also examined the immunogenicity of SFVG particles expressing HIV-1 envelope (env) or VSV nucleocapsid (N) proteins behind a second SFV promoter. These vectors unexpectedly generated strong primary CD8 T-cell responses to the foreign proteins as well as memory T-cell responses that can be recalled to high levels after boosting, without the need for long passage of the virus like particles previously expected to be required to generate a suitable level of immune response required to enable use of alphavirus VLPs in vaccine systems.

The inventors of the present invention have surprisingly discovered a hybrid viral vector system which can be used as a surprisingly effective vaccine eliciting antibody and cellular immune responses, including memory T cell responses, to any antigen of choice incorporated into the system.

Additionally, the present inventors have surprisingly shown that use of the vaccine system of the present invention leads to unexpectedly high cell mediated immune responses allowing the generation of an efficient and versatile vaccine system which enables any gene of choice to be inserted and expressed in the system.

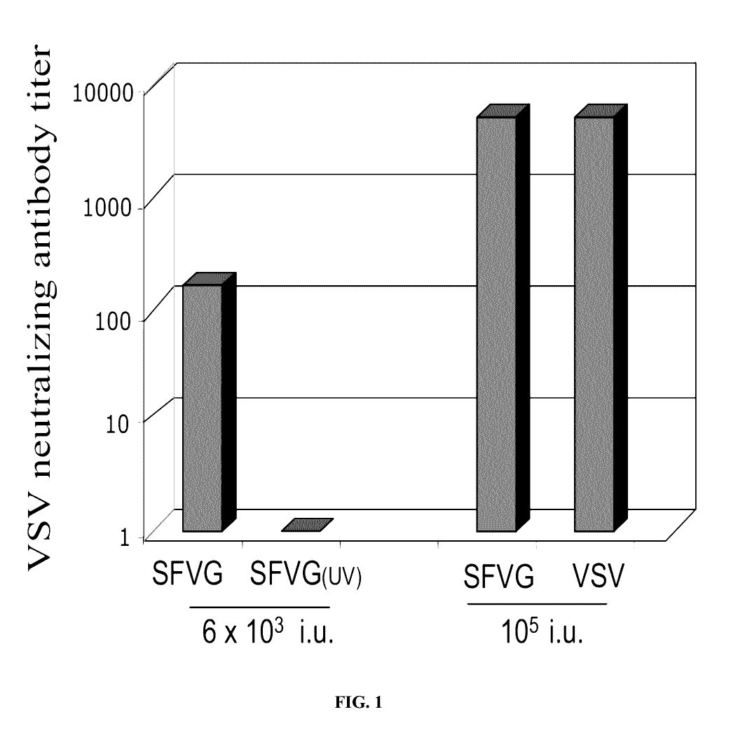

It has been surprisingly found that a relatively low dose of infectious units (10⁵ i.u.) is capable of achieving a protective neutralizing antibody titer. Remarkably, the neutralizing titer from the animals receiving 10⁵ i.u. of SFVG particles was equivalent to that generated by inoculation of the same titer of VSV (FIG. 1, Right). In an additional experiment, mice were inoculated with 2.5×10⁵ SFVG particles and no increase in the VSV-neutralizing antibody titers was observed, which surprisingly indicates that once a threshold dose is achieved, increasing the dose may have no additional effect (i.e. that efficacy was maximal at 10⁵). An advantage of the systems of the present invention is that contrary to alphavirus systems known in the prior art, the present invention has no requirement for expression of viral packaging or replicase proteins in trans. Thus, the present system simplifies alphavirus-platform vaccines. Any suitable alphavirus may be used in the construction of the vaccines of the present invention, including but not limited to Semliki forest virus, Sindbis virus, O'nyong'nyong virus, Chikungunya virus, Mayaro virus, Ross River virus, Barmah Forest virus, Eastern equine encephalitis virus, Western equine encephalitis virus and Venezuelan equine encephalitis virus.

Additionally, the first structural nucleotide sequence may be any viral envelope or capsid nucleotide sequence where the sequence encodes a protein or protein fragment used by the virus to infect a cell. Suitable examples are vesiculovirus, or rhabdovirus, structural proteins, retroviral structural proteins, and the like. Suitable rhabdovirus sequences may be used from Bovine ephemeral fever virus, rabies virus and VSV. Suitable retroviral sequences include HIV, SIV, MMLV, HTLV, RSV env proteins or protein fragments and/or gag retroviral proteins or protein fragments.

Further, by using the DNA vaccine vectors of the present invention, the inclusion of alphavirus replication genes in the vector allows amplification of vector RNA within the transfected cell, increasing infectious virus-like particle production and amplified expression of at least the second gene sequence. Experiments have demonstrated that particles derived after DNA transfection generated responses equivalent to those shown in FIG. 1 (RNA transfection).

### Vaccine

The term “vaccine” is used herein to denote to any composition containing an immunogenic determinant, for example an antigenic determinant, which stimulates the immune system such that it can better respond to subsequent infections. It will be appreciated that a vaccine usually contains an immunogenic determinant and an adjuvant, the adjuvant serving to non-specifically enhance the immune response to that immunogenic determinant. Suitable adjuvants are readily apparent to the person skilled in the art, and include Freund's complete adjuvant, Freund's incomplete adjuvant, Quil A, Detox, ISCOMs or squalene. However, it will be appreciated that the vaccine of the present invention may also be effective without an adjuvant.

The invention also provides a method for exposing an animal to a vaccine of the invention by administering a pharmaceutically acceptable quantity of the vaccine of the invention, optionally in combination with an adjuvant, sufficient to elicit an immune response in the animal.

Furthermore, the invention provides for the use of the vaccine vector in the manufacture of a medicament to increase the levels of protection of a subject against a pathogenic or cancerous agent expressing an antigen that is expressed by at least the second nucleotide sequence in the vector.

The animal is typically a human. However, the invention can also be applied to the treatment of other mammals such as horses, cattle, goats, sheep or swine, and to the treatment of birds, notably poultry such as chicken or turkeys. Preferably the microbial pathogen selected for use in a particular vaccine of the present invention causes disease or infection in the species of animal to which the vaccine is administered to, or a closely related species. The vaccines of this invention may be used as both prophylactic or therapeutic vaccines though it will be appreciated that they will be particularly useful as prophylactic vaccines due to their economy of production.

The vaccine compositions of the present invention may be administered by any suitable means, such as orally, by inhalation, transdermally or by injection and in any suitable carrier medium. However, it is preferred to administer the vaccine as an aqueous composition by injection using any suitable needle or needle-less technique.

It will be appreciated that the vaccine of the invention, including the use of VLP generated by vectors of the invention, may be applied as an initial treatment followed by one or more subsequent “booster” treatments at the same or a different dosage rate at an interval of from 1 to 26 weeks to a number of years between each treatment to provide prolonged immunisation against the pathogen.

### Antigenic Components

In accordance with the present invention, the second nucleotide sequence may encode any heterologous antigenic component. Typically, the heterologous antigenic component is a peptide fragment, polypeptide or protein. Optionally, the antigenic component should be suitable to allow a cell mediated immune response to be raised against it in the host when the vaccine is administered or shortly thereafter.

In certain embodiments the antigenic component is derived from a pathogenic organism which typically causes an infectious disease in a host. In certain embodiments, the pathogenic organism may be a prokaryotic cell, such as a gram positive or gram negative bacterium, or the pathogenic cell may be a protozoon, a parasite or a fungus, such as a yeast.

In certain embodiments, the pathogenic organism from which the antigenic component is derived may be selected from the group consisting of, but not limited to: members of the genus Escherichia, Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma.

In certain embodiments, the antigenic component may be a viral peptide. The virus from which the peptide is derived may be selected from the group consisting of, but not limited to: human immunodeficiency virus (HIV), hepatitis A virus (HAV), hepatitis B (HBV), hepatitis C (HCV), any other hepatitis-associated virus, human papillomavirus (HPV) and especially high-risk oncogenic human papillomavirus types, Kaposi's Sarcoma-Associated Herpesvirus (KSHV) (also known as Human Herpesvirus-8 (HHV-8)), Herpes Simplex virus (HSV) (any subtype), Respiratory Syncytial Virus (RSV) and associated respiratory viruses, Influenza viruses, coronaviruses including SARS-associated Coronavirus (SARS-CoV), rhinovirus, adenovirus, SIV, rotavirus, human papilloma virus, arbovirus, measles virus, polio virus, rubella virus, mumps virus, papova virus, cytomegalovirus, varicella-zoster virus, varicella virus, huntavirus and any emergent virus, in particular Ebola virus, Marburg virus, West Nile virus (WNV), St Louis Encephalitis virus (SLEV), Rift Valley Fever virus (RVFV) and other members of the Bunyaviridae.

In embodiments, the antigenic component can be derived from a protozoan pathogen, the protozoa may typically be an intracellular protozoan, such as leishmania or trypanosoma. In embodiments where the antigenic component can be derived from a yeast or fungi, said fungi may be derived from a genus selected from the group comprising: Acremonium, Alternaria, Amylomyces, Arthoderma, Aspergillus, Aureobasidium, Blastochizomyces, Botrytis, Candida, Cladosporium, Crytococcus, Dictyostelium, Emmonsia, Fusarium, Geomyces, Geotrichum, Microsporum, Neurospora, Paecilomyces, Penicillium, Pilaira, Pityrosporum, Rhizopus, Rhodotorula, Saccharomyces, Stachybotrys, Trichophyton, Trichoporon, or Yarrowia.

In certain embodiments, the antigenic component may be derived from a tumour cell. In such embodiments, typically the antigenic component is, or is a fragment of, a tumour specific antigen. In certain embodiments the tumour cell may be derived from a cancerous or malignant condition selected from the group including Acute and Chronic Myelogenous Leukemia (AML, CML), Follicular Non-Hodgkins lymphoma, malignant melanoma, Hairy Cell leukaemia, multiple myeloma, carcinoid tumours with carcinoid syndrome and liver and lymph node metastases, AIDS related Kaposi's sarcoma, renal cell carcinoma, adenocarcinoma of the large bowel, squamous cell carcinoma of the head and neck. As such antigenic components may be used in the vectors and methods of the present invention, the resultant immune response generated may be used to attack cancers and thus the vectors and viruses produced by said vectors can be designed to be oncolytic.

In accordance with the present invention, the antigenic component may be an immunogenic peptide. As used herein the term “immunogenic peptide” relates to any peptide, polypeptide, or protein fragment which is capable of eliciting an immune response in a mammal.

### DNA Vectors

As disclosed herein, DNA vectors of the present invention may be used as a vaccine in an animal, e.g. in a mammal, to elicit strong cell mediated immune responses. In order to achieve strong DNA vector expression post transfection, any suitable transfection method or method of administration of the vaccine system of the present invention may be used.

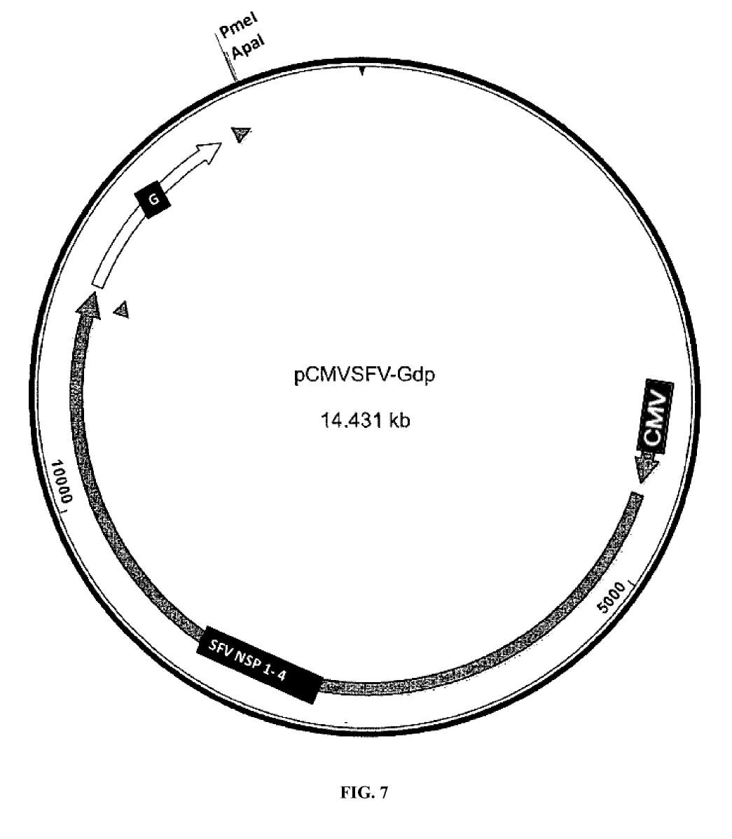

As shown in the attached FIG. 7, pCMVSFV-Gdp is a suitable DNA vector in accordance with the present invention. A nucleotide sequence encoding a second sequence encoding an antigenic component may be inserted at the Apa1 or Pme1 restriction sites as shown in FIG. 7 using standard techniques well known to the person skilled in the art. For example, the inventors used as a starting point a vector pBK-T-SFV1 (Karlsson and Liljestrom (2004)). This vector has a CMV promoter positioned to drive alphavirus (e.g. SFV) RNA production. In order to express the HIV gp140 peptide, the Apa I site in pBK-T-SFV1 was eliminated. The 4576 nucleotide Spe 1-Sac I fragment from the modified pBK-T-SFV1 vector was removed and the 6105 nucleotide Spe 1-Sac I fragment was cloned in from pSFVG-gp140. This new vector then drives expression of RNA encoding the SFV non-structural proteins, VSV G, and HIV gp140. The G and gp140 proteins are expressed from separate subgenomic mRNAs generated by the SFV RNA-dependent RNA polymerase following replication of the RNA. The sequence encoding gp140 can be removed by digestion with Apa I and other genes can be cloned in its place.

As would be understood by the person skilled in the art, the vectors of the present invention may be used in DNA or RNA form. Additionally, virus like particles generated from transfected cells may be harvested and used to infect an animal. Upon infection with the non-pathogenic VLPs, an immune response is generated to the proteins contained within the VLPs.

### Protein Expression System

As used herein, the term protein expression system includes use of the vectors of the present invention to express one or more selected nucleotide sequences in a cell. The vectors of the present invention may be modified by the inclusion of alternative protein sequences of choice. Suitable cells for use in protein expression systems include but are not limited to animal cells, for example BHK-21 (baby hamster kidney) and CHO (Chinese hamster ovary) cells. The system may also be used in e.g. insect or plant cells.

Unless otherwise defined, all technical and scientific terms used herein have the meaning commonly understood by a person who is skilled in the art in the field of the present invention.

Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers.

As used herein, terms such as “a”, “an” and “the” include singular and plural referents unless the context clearly demands otherwise. Thus, for example, reference to “an active agent” or “a pharmacologically active agent” includes a single active agent as well as two or more different active agents in combination, while references to “a carrier” includes mixtures of two or more carriers as well as a single carrier, and the like.

As used herein, the term “nucleotide sequence” refers to a length of a number of nucleotides and may refer to DNA, cDNA, or RNA. The nucleotide sequences may be constructed from natural nucleic acid bases, synthetic bases, or mixtures thereof.

The term “gene” has been used herein to refer to a polynucleotide having a nucleotide sequence which encodes a protein.

The terms “peptide”, “polypeptide” and “protein” are used herein interchangeably to describe a series of at least two amino acids covalently linked by peptide bonds or modified peptide bonds such as isosteres. No limitation is placed on the maximum number of amino acids which may comprise a peptide or protein. Furthermore, the term polypeptide extends to fragments, analogues and derivatives of a peptide, wherein said fragment, analogue or derivative retains the same biological functional activity as the peptide from which the fragment, derivative or analogue is derived.

As used herein, the term “transfection” includes any means known to the skilled man to deliver nucleotide sequences into a cell or animal. Transfection delivery methods known to the skilled man are disclosed in, but not limited to Sambrook et al. “Molecular Cloning”, A laboratory manual, cold Spring Harbor Laboratory Press, Volumes 1-3, 2001 (ISBN-0879695773). Typically transfection methods may include electroporation and lipid transfections. The skilled person is well aware of the suitable transfection methods and reagents available. For example, in the present invention in relation to DNA transfections with the DNA launched vector, the inventors transfected one microgram of DNA per million BHK cells using the LIPOFECTAMINE® (Invitrogen) transfection reagent and protocol supplied with it.

Vaccine vectors based on live viruses or viral replicons are typically potent inducers of long-lasting immune responses in animals. The Semliki Forest virus replicon has been used extensively as an effective single-cycle vaccine vector (1). This vector is normally packaged using SFV capsid protein and envelope glycoproteins expressed in trans. In the current invention, the inventors have shown that infectious membrane-enveloped particles containing VSV G protein and the SFV replicon induce potent antibody responses to the VSV G protein in mice and could protect mice from pathogenesis including lethal encephalitis caused by VSV. They also induced strong cellular immune responses to other proteins such as an HIV Env protein expressed from a second transcription unit added to the SFVG replicons.

The novel hybrid-virus vaccine platform described herein could have significant advantages over traditional alphavirus-based vectors. Because the VSV G protein is expressed directly from the replicon, there is no requirement for expression of packaging proteins in trans as in other alphavirus systems. In these complementation systems there is also the potential of reconstituting wild-type alphaviruses through recombination. Because none of the SFV structural protein genes are present in the SFVG vector, reconstitution of wild-type SFV is not possible. Also, the relatively non-specific packaging of the genomes into infectious vesicles in the absence of a nucleocapsid (8) makes it likely that there will not be a strict packaging limit for the RNA, as there is in alphaviruses or other vectors with well-defined capsid structures. The SFVG particles expressing foreign antigens can be produced in cell lines already approved for vaccine production without any requirement for modification to express complementing proteins in trans.

The inventors initially used a method of transcribing capped SFVG vector RNA in vitro and then transfecting the RNA onto cells to generate the propagating replicon particles. More recently, they have tested a DNA- launched version of the SFVG vector by using the pBK-T-SFV1 vector with a CMV promoter (1) to drive expression of the SFVG-gp140 RNA in cells. This system bypasses the in vitro transcription step and greatly simplifies production of infectious particles.

Importantly, the inventors found that the SFVG particles were nonpathogenic in mice even when given by the intravenous route, a route that allows widespread dissemination in the animal. Lack of pathogenesis probably results from inefficient particle production in the animals in the absence of a nucleocapsid protein. Pathogenic animal viruses all have capsid or nucleocapsid proteins to allow efficient packaging of their nucleic acids. This low efficiency of packaging of the particles probably prevents spread of infection from preventing significant spread from initially infected cells and rapidly limiting the infection. In one embodiment of the present invention, a G protein from a different rhabdovirus or VSV serotype or a different vesiculovirus can be used as the primary vaccine or in the boosting vector (23). There is an extensive repertoire of available vesiculovirus glycoproteins that are well known to the skilled person. Further, the G-protein based propagating replicon strategy can be extended to other alphavirus replicon systems (4) to further extend vaccine applications.

The present invention will now be described with reference to the following examples which are provided for the purpose of illustration and are not intended to be construed as being limiting on the present invention.

### EXAMPLES

### Materials and Methods

### Plasmid Construction

To construct pSFVG-gp140, a 2022 base pair (bp) DNA fragment encoding the HIVgp140 protein (IIIB strain) was amplified by PCR With VENT® polymerase (NEB) from pBSEnvG709 (24), using the forward primer 5′-GATCGATCGGGCCCAACATGAGAGTGAAGGAGAAATATC AGC-3′ (SEQ ID NO: 1), and the reverse primer 5′-ATCTGGCTACGGGCCCTC AACTTGCCC ATTTATCTAAT-3′ (SEQ ID NO: 2). Both of the primers contained an ApaI site. The PCR product was digested with ApaI, purified, and ligated into the pSFV1-Gdp vector linearized with ApaI (18). The correct sequence of the gp140-insert was verified (Yale Keck Facility).

### Transcription of RNA and Transfection to Generate Infectious Particles

To generate the infectious RNA genome of the propagating replicons, pSFV1-G and pSFVG-gp140 plasmids were linearized with Spe1, and transcribed for 2 hours at 37° C. in a 40 μl reaction mixture. The reaction was a modification of the AMPLISCRIBE™ SP6 transcription kit (Epicentre technologies) containing SP6 reaction buffer, 5 mM each of ATP, CTP, and UTP, 1 mM GTP, 4 mM m⁷G(ppp)G RNA cap analog (NEB S1404L), 10 mM DTT, and 2 μl of SP6 polymerase. The transcription reactions were stored at −80° C.

Transfection of cells for growing stocks of propagating replicons was performed as follows: 4×10⁶ BHK-21 cells were plated the day before transfection on 10 cm diameter plates. They were then transfected with 60 pi of transcription reaction in 9 ml of serum-free DMEM containing 90 ul of a cationic liposome reagent containing dimethy-dioctadecyl ammonium bromide (25) as described. (8). The cells were scraped into the medium at 28 hours post transfection and sonicated by using a Branson 450 sonicator to release infectious particles. After sonication cell debris was removed by centrifugation for 8 min at 2000 rpm in a Fisher MARATHON® table-top centrifuge, and the supernatant was transferred to new tubes. For concentration of the stocks, supernatants were transferred to Beckman ultraclear tubes and centrifuged at 40,000 rpm for 1 hour in a Beckman an SW50.1 rotor. The infectious particles were resuspended in a volume of PBS that concentrated the particles 40-fold.

### Immunofluorescence Microscopy

For titration of the stocks, BHK-21 cells plated on coverslips were infected for 21 hours with different dilutions of the virus, fixed with 3% paraformaldehyde, and incubated with a 1:200 dilution of monoclonal antibodies (26) to VSV G protein, followed by ALEXA FLUOR® 488 goat anti-mouse IgG (H+L) (Invitrogen) diluted 1:250. Green fluorescent areas of infected cells or plaques were counted on an Olympus CK40 microscope equipped with a ×10 objective, and titers were calculated. Infectious particle titers in the range of 1-5x10⁷per ml were obtained, depending on the construct. For visualization of both VSV-G and HIV 140 proteins after transfection with pSFVG-gp140 or infection with SFVG-gp140 particles, BHK-21 cells were fixed and incubated with anti VSV-G antibody as above, followed by ALEXA FLUOR® 594-conjugated goat anti-mouse IgG (1:500) secondary antibody. The cells were then permeabilized with 1% Triton-X100, and incubated with a 1:100 dilution of polyclonal sheep anti-HIVgp120 antiserum (NIH AIDS Research and Reference Reagent Program) followed by incubation with FITC-conjugated donkey anti-sheep serum diluted 1:50. Cells were observed with a Nikon ECLIPSE™ 80i fluorescence microscope equipped with a Nikon Plan Apochromat 60× oil objective and a Photometrics COOLSNAP™ camera.

### [³⁵S]-Methionine Labeling, Immunoprecipitation, and SDS/PAGE

BHK-21 cells grown to about 50% confluency on 35 mm diameter plates were infected with SFVG or SFVG-140 particles at a multiplicity of infection (m.o.i) of one. Infected cells were incubated at 37° C. for 5 hours. The medium was removed, and cells were washed twice with methionine-free Dulbecco's modified Eagle's medium (DMEM). Then 1 ml of methionine-free DMEM containing 100 pCi (1 Ci=37 GBq) of [³⁵S]-methionine was added to each plate for 1 hour at 37° C. To prepare labeled cell extracts, the medium was removed, and the cells were washed twice with phosphate buffered saline (PBS) and lysed in 500 |jl of detergent solution (1% Nonidet P-40, 0.4% deoxycholate, 50 mM EDTA, 10 mM Tris-HCl, pH 7.8) on ice for 5 min. The cell lysates were collected into 1.5 ml Eppendorf tubes and cell debris was removed by centrifugation for two minutes at 13,000 rpm.

Immunoprecipitation of VSVG and HIVgp140 proteins from the labeled cell lysates was carried out as follows. The lysates were incubated with polyclonal rabbit anti-VSV serum or a polyclonal sheep anti-HIV gp120 serum for 1 hour at 37° C. Protein A-Sepharose (Zymed Laboratories Inc., San Francisco, Calif.) was added and samples were then incubated for 30 min at 37° C. The sepharose was washed three times with radioimmune precipitation assay (RIPA) buffer (1% Nonidet P-40, 1% deoxycholate, 0.1% sodium dodecyl sulfate (SDS), 150 mM NaCl, 10 mM Tris-HCl, pH 7.8). Labeled immunoprecipitated proteins were analyzed by electrophoresis on an SDS-10% polyacrylamide gel.

### Inoculation of Mice

Eight-week-old female BALB/c and C57BL/6 mice were obtained from Jackson Laboratories and kept for at least one week before experiments were initiated. Mice were housed in microisolator cages in a biosafety level 2 equipped animal facility. Viral stocks were diluted to appropriate titers in serum-free DMEM. Mice were vaccinated by i.m. injection in the right hind leg with VSV or SFVG in a total volume of 50 pi in the back hind leg muscle. Vaccinia boosts were performed via the intraperitoneal route of infection with 1×10⁵ pfu of virus. The Institutional Animal Care and Use Committee of Yale University approved of all animal experiments done in this study.

### VSV Neutralization Assay

Blood was obtained from mice at 28 days after vaccination. Serum was collected and heat inactivated and neutralization assays were performed as described previously (23). Mouse sera were serially diluted on 96-well plates, and incubated with 100 pfu of VSV per well for 1 hour at 37° C. BHK-21 cells were added to each well, and plates were incubated at 37° C. for 2 to 3 days. Each assay was performed in duplicate. Neutralization titers are given as the highest dilutions that showed complete inhibition of VSV infection and cytopathic effect.

### Immunizations and VSV Challenge

For challenge experiments, 12 BALB/c mice were immunized with 5×10⁵ pfu of SFVG particles by i.m. injection in the right hind leg in a total volume of 50 pi. At 36 days post-immunization, these mice were challenged with 5×10⁷ pfu of wild-type VSV (Indiana serotype, San Juan strain) by the intravenous route in a total volume of 100 |jl per mouse. Twelve naive BALB/c mice were also challenged as controls. Viral stocks were diluted to appropriate titers by using serum-free DMEM. Following challenge, mice were weighed daily for up to 14 days and observed for signs of pathogenesis for a total of 60 days. Any animals exhibiting paralysis or distress during this period were euthanized.

### Tetramer Assay

The tetramer assay was performed on fresh splenocytes as described previously (27). Splenocytes were obtained seven days after the primary vaccination in all experiments and on day 35 after primary vaccination (day 6 post boost) in boosting experiments. Responses to HIV Env were measured in BALB/c mice using the Env tetramer (MHC class I D^{d}) previously described and containing the Env peptide N-RGPGRAFVTI-C (SEQ ID NO: 3) (16). Responses to VSV N were measured in vaccinated C57BL/6 mice by using the N tetramer (MHC class I Kb) previously described and containing the N peptide N-RGYVYQGL-C (SEQ ID NO: 4) (20, 21). Tetramers were obtained from the National Institute of Allergy and Infectious Diseases (NIAID) Tetramer Facility. Cells that were tetramer+, activated (CD62L¹⁰), and CD8⁺ were identified using flow cytometry as previously described (27). To determine background levels of tetramer binding, splenocytes from naive (N tetramer assay) or VSV vector vaccinated mice (Env tetramer assay) were used.

### Statistical Analysis

GraphPad Prizm software, version 4.0 was used for all analyses.

### Example 1

### Induction of Neutralizing Antibodies to VSV G Protein in Mice Inoculated With SFVG Particles Requires Vector Replication

To determine whether the propagating replicon particles were able to induce antibody responses to VSV G protein in animals, and if replication was required for such induction, mice were inoculated by the intramuscular (i.m.) route with 6×10³ infectious units (i.u.) of SFVG particles which were either untreated or inactivated with UV light to prevent RNA replication. After one month, serum neutralizing antibody titers to VSV were determined (FIG. 1 Left). These results showed 100% neutralization of VSV at serum dilutions of 1:160 for SFVG particles, but no detectable neutralizing Ab (<1:20) in animals given the UV-inactivated particles. These results indicate that the incoming G protein on the particles was not present in sufficient amounts to induce VSV neutralizing antibody, and that G protein must be synthesized in infected cells to generate such responses.

The inventors next determined if the strength of the antibody response to VSV G was dose-dependent. Groups of three mice were inoculated with 10⁵ i.u. of SFVG particles or with 10⁵ plaque forming units (pfu) of VSV. VSV serum neutralizing titers were determined at 28 days after infection by using pooled serum from each group.

The neutralizing antibody responses to VSV in sera from mice inoculated with the ‘high’ dose (10⁵ i.u.) of SFVG was 1:5,120, 32-fold higher than that induced in mice inoculated with the ‘low’ dose (6×10³ i.u.) of SFVG. Remarkably, the neutralizing titer from the animals receiving 10⁵ i.u. of SFVG particles was equivalent to that generated by inoculation of the same titer of VSV (FIG. 1, Right). In an additional experiment, mice were inoculated mice with 2.5×10⁵ SFVG particles and found no increase in the VSV-neutralizing antibody titers was observed.

### Example 2

### Vaccination with SFVG Particles Protects Mice From Pathogenesis Following VSV Challenge

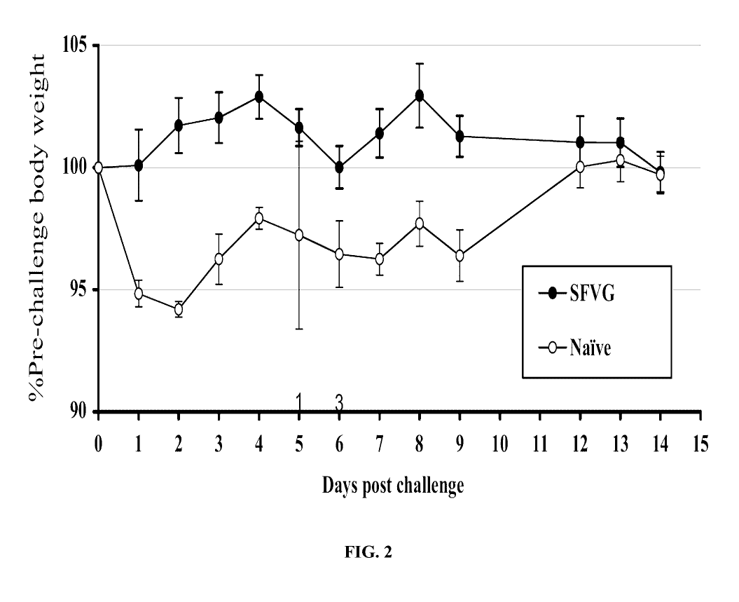

Intravenous injection of wild-type VSV in BALB/c mice causes severe weight loss over 4-5 days and also causes lethal encephalitis in 20-40% of the animals. To determine if immunization with SFVG particles was sufficient to protect mice from such pathogenesis, 12 mice were immunized i.m. with SFVG particles and then challenged 36 days later with 5×10⁷ pfu of wild-type VSV by the intravenous route. Following challenge, the mice were weighed daily to follow pathogenesis. The SFVG-immunized mice maintained the same or higher than pre-challenge body weights following challenge and showed no signs of pathogenesis (FIG. 2). In contrast, all 12 age-matched naive control mice showed dramatic weight loss following the identical challenge, along with other signs of pathogenesis including ruffled fur and hunched posture. In addition 4/12 of the control mice developed the severe hind-limb paralysis indicative of VSV encephalitis and died or were euthanized on days 5 and 6. There was a significant difference (p=<0.05, Mann-Whitney test) in weight loss between the SFVG immunized group and the control group, through day 7 post challenge. After day seven, the remaining animals in the control group began to recover to normal weight. The protection from paralysis (encephalitis) was also statistically significant (p=0.047, Fisher's exact test) between the immunized and control groups.

We also checked VSV neutralizing antibody titers were checked in individual immunized animals at day 30, six days prior to challenge. They ranged from 1:640 to 1:5120 in the twelve vaccinated animals. The control animals had undetectable VSV neutralizing antibody titers. The high titer antibodies in the vaccinated animals are consistent with the complete protection observed.

### Example 3

### SFVG Replicon Particles are not Pathogenic in Mice

After i.m. injections of SFVG particles, there were now had not seen any signs of pathogenesis in mice. To determine if there was any detectable pathogenesis caused by infection by other potentially more pathogenic routes, we gave the SFVG particles were administered by both the intravenous and intranasal routes (10⁵ i.u.). The mice were then weighed the mice daily for two weeks and then observed the mice for 60 days: and saw no signs of pathogenesis caused by the particles was observed.

### Example 4

### Generation of SFVG Replicons Expressing HIVgp140

To evaluate the ability of infectious SFVG particles to generate cell-mediated immune responses, we generated particles expressing the HIV-1 (IIIB) gp 140 gene were generated. This gene encodes a secreted form of HIV Env protein lacking the transmembrane and cytoplasmic portions of gp41 (14). There is an immunodominant CD8 T cell (p18) epitope (15, 18) in this gp140 protein (BALB/c mice), and the inventors used an MHC I tetramer that recognizes T cells specific for this epitope, allowing precise quantitation of the CD8 T cell response (17). The gp140 gene was inserted into the pSFVdpG-X vector (18) downstream from a second SFV promoter. To generate the replicons, RNA transcribed in vitro from this vector was used to transfect BHK-21 cells, and infectious particles were recovered after 28 hours as described in Materials and Methods. Infectious SFVG-gp140 particles derived from pSFVdpG-gp140 were expected to encode VSV G and HIV gp140 from separate mRNAs.

### Example 5

### Coexpression of VSV G and HIVgp140 Proteins in Infected Cells

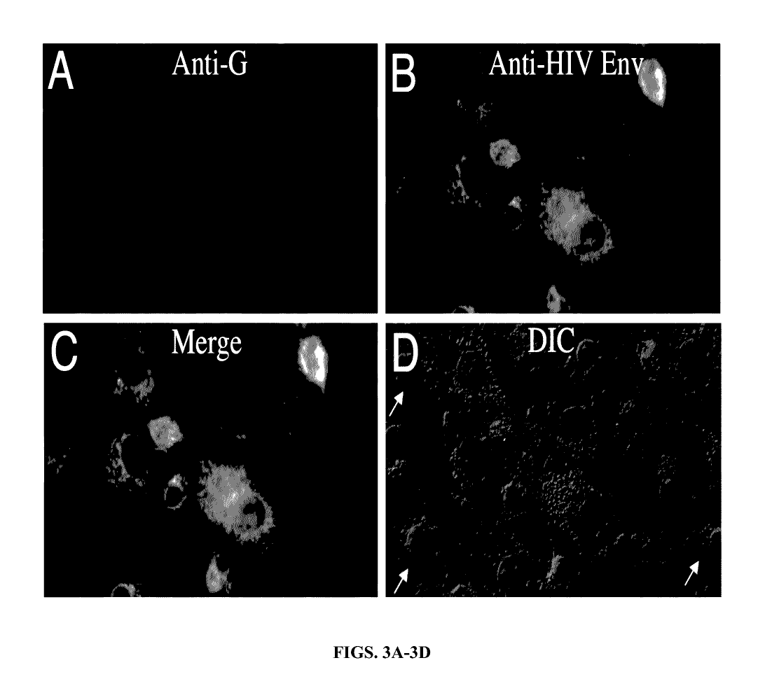

To determine if the SFVG-gp140 particles expressed both VSV G and gp140 proteins, BHK-21 cells were infected with these particles for 20 hours. Cells were then fixed and expression of both gp140 and VSV G was detected by indirect immunofluorescence (FIGS. 3A-3D). VSV G protein was expressed predominantly on the cell surface (red, FIG. 3A) while HIVgp140 was expressed in a pattern typical of the endoplasmic reticulum (green, FIG. 3B) in a focus of infection. The merged image (FIG. 3C) shows that cells expressing VSV G also expressed HIV gp140. The DIC (differential interference contrast) image of the same field shows that some cells in the periphery of the focus of infection (white arrows) were not yet infected and expressed neither G nor gp140 (FIG. 3D).

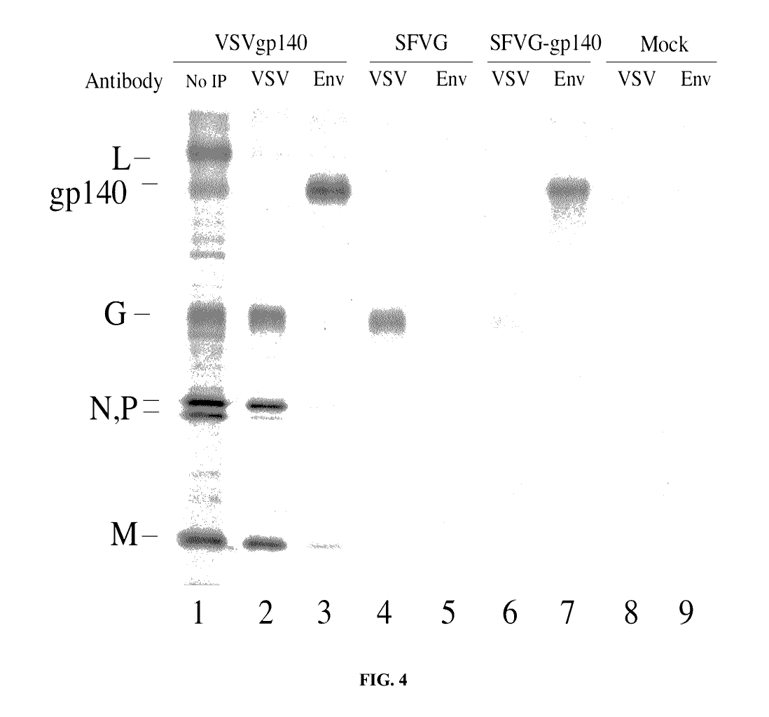

For direct visualization of the sizes of proteins expressed by the SFVG-gp 140 particles metabolic labeling of infected cells with (7)-methionine was performed. BHK-21 cells were infected with SFVG, SFVG-gp140 or with a VSV recombinant expressing gp140 (14) and labeled with [³⁵S]-methionine for one hour. Cell lysates were prepared and either fractionated directly by SDS-PAGE or immunoprecipitated using antibodies to VSV or HIV Env prior to PAGE as indicated (FIG. 4). VSV proteins (L, G, N, P, M) as well as gp140 are easily seen without immunoprecipitation because of the effective shut off of host protein synthesis (lane 1). Anti-VSV antibody precipitated all VSV proteins (lane 2), whereas anti-Env antibody precipitated gp140 (lane 3) from the VSVgp140 lysate. Cells infected with SFVG particles expressed VSV G but not gp140 (lanes 4 and 5) while cells infected with SFVG-gp140 expressed both G and gp140 (lanes 6 and 7). Mock infected cells were used as controls (lanes 8 and 9).

### Example 6

### SFVG-gp140 Particles Elicit Env-Specific CD8 T Cell Responses

To determine if the SFVG vector expressing HIV Env gp140 was able to induce a CD8 T cell responses to Env, mice were vaccinated i.m. with SFVG-gp140 particles and an MHC I tetramer assay was used, employing an H-2 D^{d} tetramer loaded with the immunodominant peptide p18-110 (16) from HIV 1Mb Env protein (17).

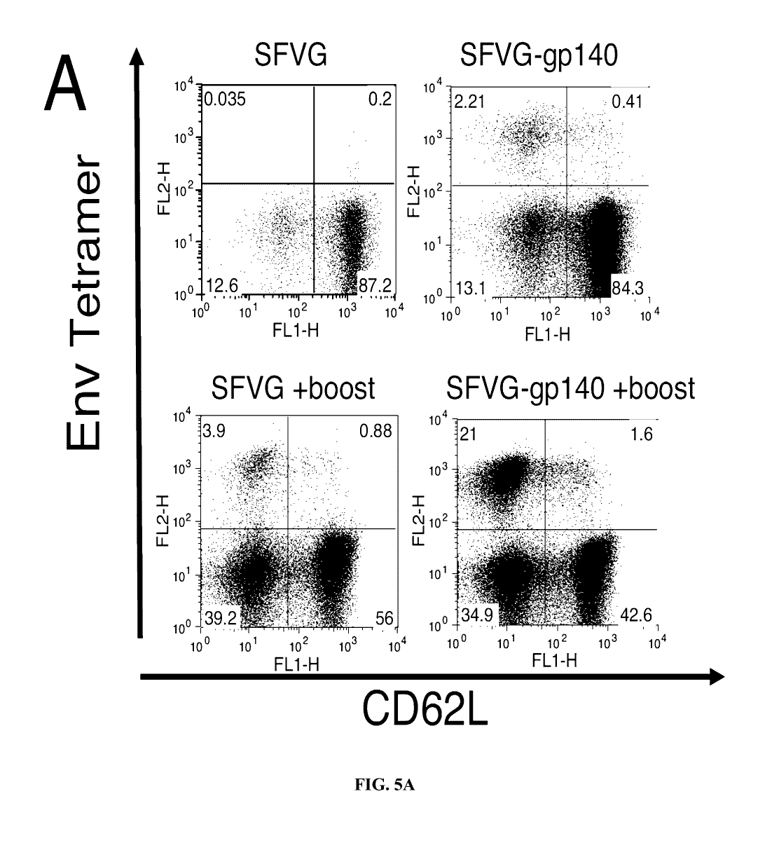

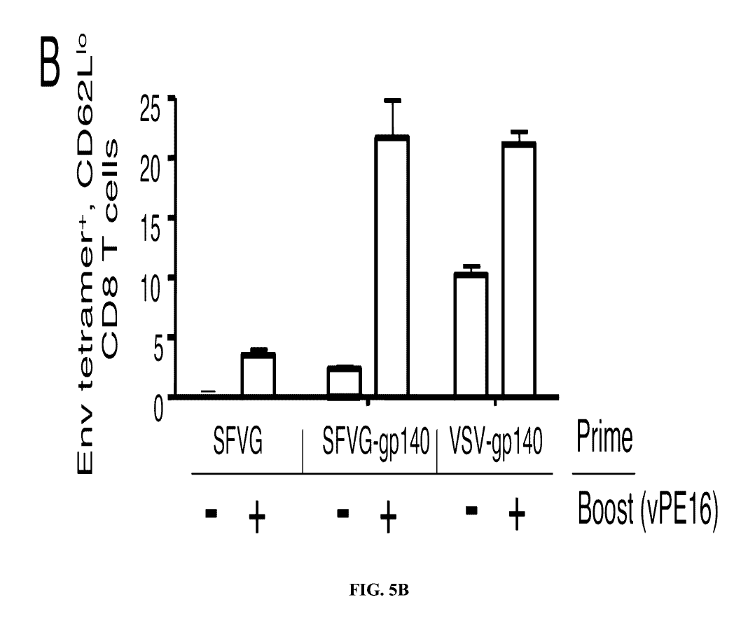

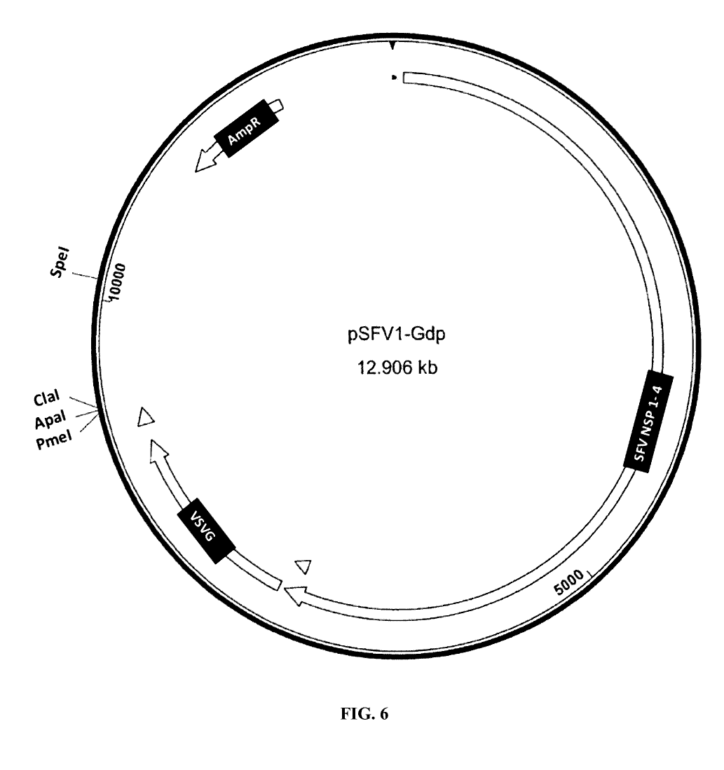

Seven days after vaccination with SFVG-gp140, mice had a substantial population of activated Env-specific CD8 T cells (2.3%±0.3% CD62L¹⁰, tetramer⁺, CD8 T cells, FIGS. 5A-5B). The population elicited by SFVG-gp140 was similar to the primary response elicited by the vaccinia vectors (3.5%/±0.5%, CD62L¹⁰, tetramer⁺ CD8 T cells) in mice that had previously seen only the control SFVG vector (FIGS. 5A-5B) or in naive mice (<0.06%). This response is also the same as that generated in naive mice given the vaccinia vector expressing HIV Env (17). The primary response to Env elicited by SFVG-gp140 was 4- to 5-fold lower than that elicited by VSV-gp140 (FIG. 5B).

Due to the ability of SFVG-gp140 particles to elicit a strong primary T-cell response, the recall of memory cells after a boost with vaccinia expressing HIV Env was examined. On day 29 post prime, mice were boosted with vaccinia virus (vPE 16) expressing the HIV Env protein (19). Recall, Env- specific CD8 T cell responses were measured 6 days post boost at day 35. Mice primed with VSVgp140 or SFVG-gp140 elicited a strong recall response after vaccinia boost. In fact, the Env-specific CD8 T cell response was equivalent post boost when primed with either the VSV or the SFV vector (FIG. 5B; 21.2% ±1.1% and 21.6%±3.2%, respectively).

To examine the versatility of the vector system of the present invention, an SFVG-N vector that expresses the VSV nucleocapsid protein (18) was also tested and its ability to initiate cellular immune responses was analyzed. Mice were vaccinated i.m. with SFVG-N and looked at the primary and recall responses to VSV N. For these experiments we used an H-2K^{b} tetramer containing an immunodominant peptide from VSV N (20, 21). The inventors found a defined population of N-specific CD8 T-cells in the spleens of animals vaccinated with SFVG-N (0.5% CD62L¹⁰, tetramer+) which were boosted to high levels (12% CD62L¹⁰, tetramer+) with a vaccinia recombinant (v38 (22)) expressing VSV N protein.

To examine the versatility of the vector system of the present invention, an SFVG-N vector that expresses the VSV nucleocapsid protein (18) was also tested and its ability to initiate cellular immune responses was analyzed. Mice were vaccinated i.m. with SFVG-N and looked at the primary and recall responses to VSV N was observed. For these experiments we used an H-2K^{b} tetramer containing an immunodominant peptide from VSV N was used (20, 21).

The inventors found a defined population of N-specific CD8 T-cells in the spleens of animals vaccinated with SFVG-N (0.5% CD62L¹⁰, tetramer+) which were boosted to high levels (12% CD62L¹⁰, tetramer+) with a vaccinia recombinant (v38 (22)) expressing VSV N protein.

All documents referred to in this specification are herein incorporated by reference. Various modifications and variations to the described embodiments of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

### REFERENCES

Karlsson G B, Liljestrom P (2004) Methods Mol Biol 246:543-557.
Olkkonen V M, et al. Methods Cell Biol 43(Pt A):43-53.
Pushko P, et al. (1997) Virology 239:389-401.
Rice C M (1996). Adv Exp Med Biol 397:31-40.
Liljestrom P, Garoff H (1991) Bio/Technology 9:1356-1361.
Rolls M M, et al. Cell 79:497-506.
Florkiewicz R Z Rose J K (1984) Science 225:721-723.
Riedel H et al., (1984). EMBO J 3:1477-1483.
Kelley J M, et al. (1972). J Virol 10:1231-1235.
Salonen A et al., (2005) Curr Top Microbiol Immunol 285:139-173.
Froshauer S et al. (1988) J Cell Biol 107:2075-2086.
Grimley P M et al. (1968) J Virol 2:1326-1338.
Kujala P, et al. (2001) J Virol 75:3873-3884.
Publicover J et al. (2005) J Virol 79:13231-13238.
Shirai M et al.(1997) J Immunol 158:3181-3188.
Shirai M et al. (1992) J Immunol 148:1657-1667.
Haglund K, et al. (2002) J Virol 76:2730-2738.
Rolls M M et al. (1996) Virology 218:406-411.
Earl P L et al. (1991) J Virol 65:2047-2055.
Masopust D et al. (2001) Science 291:2413-2417.
Van Bleek G M et al. (1990) Nature 348:213-216.
Mackett M et al. (1985) Science 227:433-435.
Rose N F et al. (2000) J Virol 74:10903-10910.
Owens R J, Rose J K (1993) J Virol 67:360-365.
Rose J K et al. (1991) BioTechniques 10:520-525.
Lefrancios L et al. (1982) Virology 121:157-167.
Publicover J et al. (2004) J Virol 78:9317-9324.

1-50. (canceled)

51. An RNA replicon particle generated by a DNA hybrid-virus vector vaccine comprising:
a Semliki Forest virus (SFV) non-structural protein nucleotide sequence;
a first nucleotide sequence comprising a vesicular stomatitis virus (VSV) envelope spike glycoprotein (G protein) sequence (VSVG);
a second nucleotide sequence that encodes a heterologous antigen protein;
wherein the vector lacks functional nucleotide sequences which encode a SFV nucleocapsid protein and at least one spike protein;
and further wherein the DNA hybrid-virus vector comprises a cytomegalovirus (CMV) immediate early promoter.

52. The RNA replicon particle of claim 51, wherein the VSVG nucleotide sequence is operably linked to a SFV subgenomic promoter.

53. The RNA replicon particle of claim 51, wherein the heterologous antigen protein is expressed on the surface of the RNA replicon.

54. The RNA replicon particle of claim 51, wherein the heterologous antigen protein is a viral protein or fragment thereof.

55. The replicon particle of claim 54, wherein the viral protein or fragment thereof is derived from SIV, HIV-1 or HIV-2.

56. The replicon particle of claim 51, wherein the DNA hybrid-virus vector vaccine comprises a third nucleotide sequence wherein the third nucleotide sequence encodes a further heterologous antigen protein.

57. A vaccine composition comprising the RNA replicon particle of claim 51.

58. The vaccine composition of claim 57, wherein the RNA replicon particle is non-pathogenic to a cell or an animal.

59. A pharmaceutical composition comprising the RNA replicon particle of claim 51 and at least one pharmaceutically acceptable carrier, diluent or excipient.

60. The pharmaceutical composition of claim 59, further comprising at least one adjuvant.

61. The pharmaceutical composition of claim 60, wherein the adjuvant is selected from the group consisting of Freund's complete adjuvant, Freund's incomplete adjuvant, Quil A, Detox, ISCOMs and squalene.

62. An RNA replicon particle generated by a protein expression system comprising a DNA hybrid-virus vector vaccine comprising:
a Semliki Forest virus (SFV) non-structural protein nucleotide sequence;
a first nucleotide sequence comprising a vesicular stomatitis virus (VSV) envelope spike glycoprotein (G protein) sequence (VSVG);
a second nucleotide sequence that encodes a heterologous antigenic protein;
wherein the vector lacks functional nucleotide sequences which encode a SFV nucleocapsid protein and at least one spike protein;
and further wherein the DNA hybrid-virus vector comprises a cytomegalovirus (CMV) immediate early promoter.


SEQUENCE LISTING (4 sequences)

SEQ ID NO: 1 - DNA, 42 bp, Artificial Sequence
  Primer
gatcgatcgggcccaacatgagagtgaaggagaaatatcagc

SEQ ID NO: 2 - DNA, 38 bp, Artificial Sequence
  Primer
atctggctacgggccctcaacttgcccatttatctaat

SEQ ID NO: 3 - protein, 10 aa, Human immunodeficiency virus
RGPGRAFVTI

SEQ ID NO: 4 - protein, 8 aa, Vesicular stomatitis virus
RGYVYQGL